(19) United States
(12) Patent Application Publication
(10) Pub. No.: US 2015/0135544 A1
(43) Pub. Date: May 21, 2015

(54) Ride Stick Poster

(71) Applicants: Everett Albert Jesse, Carson City, NV (US); Tamera Heller Jesse, Carson City, NV (US)

(72) Inventors: Everett Albert Jesse, Carson City, NV (US); Tamera Heller Jesse, Carson City, NV (US)

(21) Appl. No.: 14/086,961

(22) Filed: Nov. 21, 2013

(51) Int. Cl. A61B 5/107; G01B 1/00

(52) U.S. Cl. CPC A61B 5/1072; G01B 1/00
USPC 33/493; 33/494

(57) ABSTRACT

The Ride Stick Poster combines a picture, image and or art with a straight edge ruler printed, or placed, vertically on top of the background picture, image and or art, The horizontal lines that make up the straight edge ruler may obscure the background picture, image and or art but in an aesthetically pleasing way, usually printed or placed to one or more sides of the poster. Notes are printed or placed on the poster at or near the height requirements of various rides and or attractions for a particular amusement park, theme park or fair. Notes including ride and or attraction heights, trademarks, copyrights, logos, sales and marketing information, and or other information can be displayed on top of the background picture, image and or art. The notes should be placed on the poster so as not to obscure the straight edge ruler(s) and in a location that makes the poster aesthetically pleasing. The straight edge ruler, or rulers, do not have to start at zero. For example the measurement at the bottom of the poster may start at 24 inches (61 centimeters) and at the top of the poster end at 76 inches (193 centimeters).

### CROSS REFERENCE TO RELATED APPLICATIONS

Not Applicable

### STATEMENT REGARDING FEDERALLY SPONSORED RESEARCH OR DEVELOPMENT

Not Applicable

### PARTIES TO A JOINT RESEARCH AGREEMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

As a person or pet grows, height is often recorded on a door jam or on a wall or some other permanent fixture in order to see changes over time. A tape type measuring device or straight edge ruler, or measuring stick, is not conducive to hang on a wall as a permanent record of a person's height due to the fact that these measuring devices are not aesthetically pleasing in a typical home. Medical devices that are used to measure height, Stadiometers or height rods, are accurate and calibrated devices, but they too are not aesthetically pleasing in a typical home and are usually quite expensive when compared to tape or straight edge ruler measuring devices.

Stadiometers, also known as height rods, are a quick, easy and accurate way to measure height for both adults and children. Stadiometers, or height rods, are usually found in doctors offices, clinics, medical research facilities, health facilities, schools, universities and hospitals. These height measuring devices can be wall mounted or can be self supported. Sometimes these height measuring devices can be designed for infants, small children and pets, and they can also be portable. Stadiometers, or height rods, can have digital readouts that help improve height measurement accuracy.

A height backdrop chart can be used to quickly gage the height of an individual or thing. Height backdrop charts are often used in police stations as the backdrop for mug shots and line ups of potential criminals. These charts usually have a white background with black horizontal lines indicating height. A person just stands in front of the height backdrop chart and one can determine their approximate height. These are also used at the exits of convenience stores, gas stations, restaurants and banks to determine the height of customers that use the facilities.

People display and or hang posters, pictures, art and images on their walls at home, work, schools, public and private buildings, churches, meeting halls, and public, private and non-profit businesses.

People go to amusement parks, theme parks and fairs to ride various rides or attractions. Often times there are height requirement to go on a particular ride or attraction. The height requirements may be listed in advertising, printed material, on signs, the Internet or at the ride entrance. A person has to know their height and compare it to the ride or attraction's listed height to see if they are tall enough to go on the ride. The person may also go to the park or fair, then go to the ride or attraction, and then determine if they qualify to participate on the ride or attraction by standing against a measuring gauge, indicator or device. Sometimes the person is not tall enough to go on the ride or attraction.

### SUMMARY OF THE INVENTION

The Ride Stick Poster combines a picture, image and or art with a straight edge ruler printed, or placed, vertically on top of the background picture, image and or art. The horizontal lines that make up the straight edge ruler may obscure the background picture, image and or art but in an aesthetically pleasing way, usually printed or placed to one or more sides of the poster. Notes can be printed or placed on the picture, image and or art at or near the height requirements of various rides or attractions for a particular amusement park, theme park or fair. Notes can also be printed or placed on the picture, image and or art as a form of advertisement and or marketing, Notes including ride and or attraction heights, trademarks, copyrights, logos, sates and marketing information, and or other information can be displayed on top of the background picture, image and or art. The notes should be placed on the poster so as not to obscure the straight edge ruler, or rulers, and in a location that makes the poster aesthetically pleasing. The straight edge ruler, or rulers, do not have to start at zero. For example the measurement at the bottom of the poster may start at 24 inches (61 centimeters) and at the top of the poster end at 76 inches (193 centimeters).

### DESCRIPTION OF THE DRAWINGS

FIG. 1 Is a plan view of the Ride Stick Poster with ride and or attraction height(s) and other notes.

FIG. 2 Is a wider version of FIG. 1, and is also a plan view of the Ride Stick Poster with ride and or attraction height(s) and other notes.

FIG. 3 Shows the thickness of the Ride Stick Poster.

FIG. 4 Is an exploded view of a segment of the Ride Stick Poster with ride and or attraction height(s) and other notes.

FIG. 5 Is the back view of the Ride Stick Poster with associated notes, ride and or attraction height(s), instructions and marketing information.

FIG. 6 Is a wider version of FIG. 5, and is also the back view of the Ride Stick Poster with associated notes, ride and or attraction height(s), instructions and marketing information.

### DETAILED DESCRIPTION OF THE INVENTION

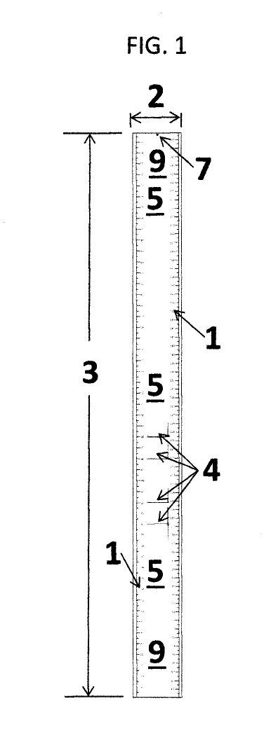

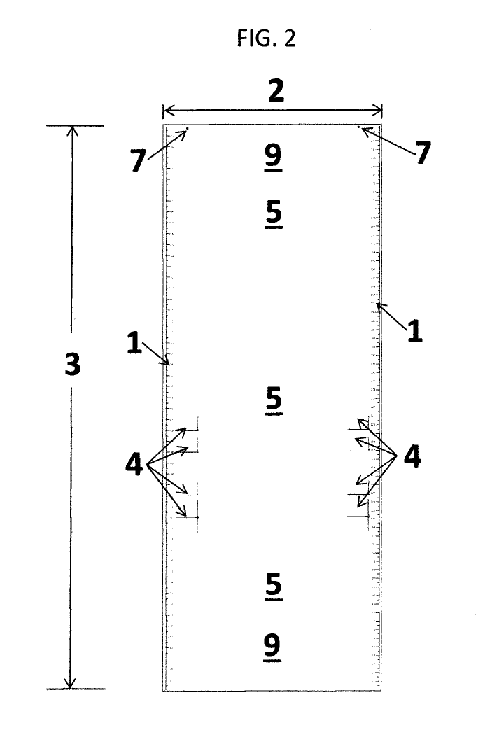

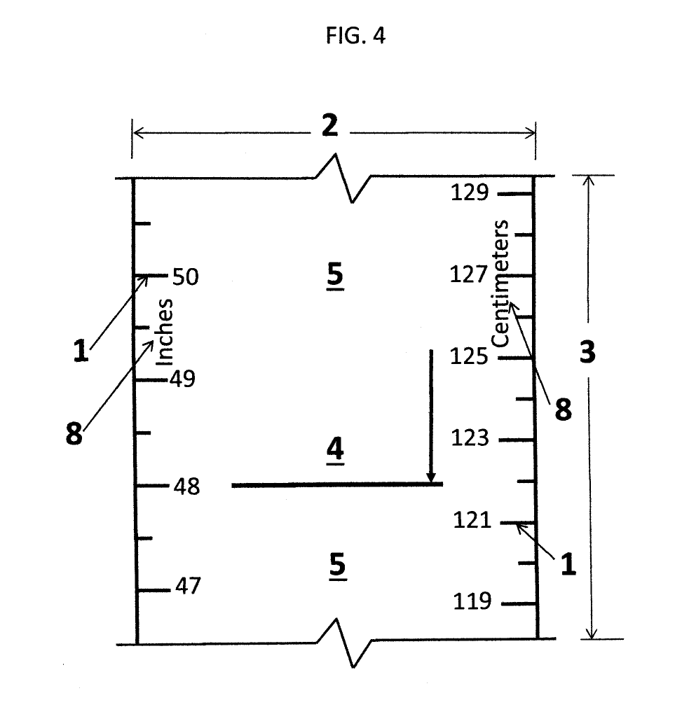

The Ride Stick Poster combines a background picture, image and or art with a straight edge ruler printed or placed on top of the picture, image and or art. Reference Number 5 on FIG. 1, FIG. 2 and FIG. 4 is the background picture, image or art. Reference Number 1 on FIG. 1, FIG. 2 and FIG. 4 is an example of the straight edge ruler(s). The straight edge ruler on the Ride Stick Poster can be printed using inches, fractions of an inch, tenths of inches, centimeters and or millimeters. Other units of measure, like feet, meters, and or fractions thereof can also be used. The units of measurement height, and in some cases width, are not limited.

FIG. 1, FIG. 2 and FIG. 4 Reference Number 1 shows the typical configuration for the straight edge ruler displayed vertically on both sides of the poster. The straight edge ruler on the left of the figure is in inches, with half inch horizontal marks and every inch noted, and on the right of the poster the measurement is in centimeters, with every two centimeters noted. FIG. 4 shows an exploded view of a segment of the straight edge ruler on the left side in inches and on the right side in centimeters. The straight edge ruler is displayed vertically on top of the background picture, image and or art so as not to obscure the picture, image and or art, or if the straight edge ruler does obscure a portion of the poster it is done in an aesthetically pleasing way. The examples shown in FIG. 1, FIG. 2 and FIG. 4 show two measurement scales one in inches on the left and the other in centimeters on the right. The poster can have one, or two or more straight edge rulers displayed vertically. The poster can also have one or more straight edge ruler(s) going horizontally, this is especially useful for measuring and noting width. The straight edge ruler measurement, see FIG. 1, FIG. 2 and FIG. 4 Reference Number 1, does not have to start at zero. For example the measurement at the bottom of the poster may start at 24 inches (61 centimeters) and at the top of the poster end at 76 inches (193 centimeters). The straight edge measurement is accurate to +/−0.1 inches or +/−0.25 centimeters. The Ride Stick Poster is novelty item and is not a calibrated medical or measurement device.

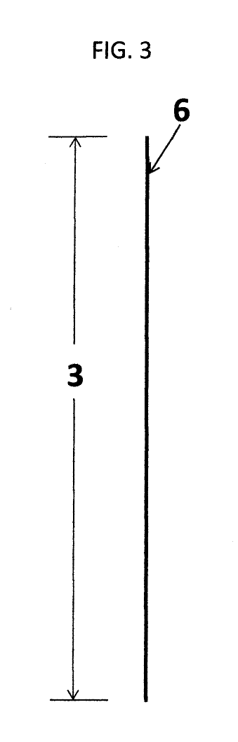

The Ride Stick Poster backing can be made from paper, plastic, metal, textile material or other material or combination of materials. The thickness of the poster backing material, FIG. 3-6 Reference Number 6, can vary usually from 0.0005 to 1.5 inches (0.012 to 38 millimeters) and is dependent upon the materials or combination of materials used. The poster backing material is preferably made from paper or plastic usually 0.02 inches (0.5 millimeters) thick. Regardless of the material used it should lay flat when displayed on a wall, or when hung from hooks or rings, and can be rolled up for ease of sales and marketing or laid flat for shipping. The Ride Stick Poster may be produced with pre-punched holes for ease of hanging on a wall or display, see FIG. 1 and FIG. 2 Reference Number 7.

Displayed, or printed, on top of the poster backing material can be any picture, image and or art. The background picture, image and or art are not limited. The straight edge ruler, or rulers, can be displayed in any color or combinations of colors, including white and black, so as to make the ruler(s) visible with respect to the background picture, image and or art.

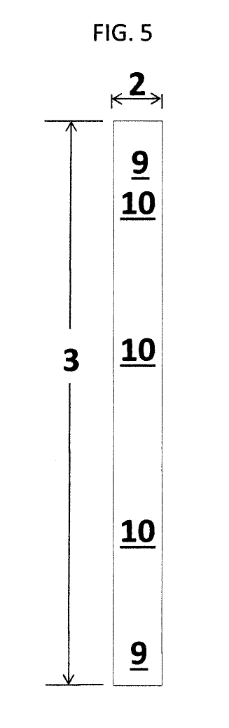

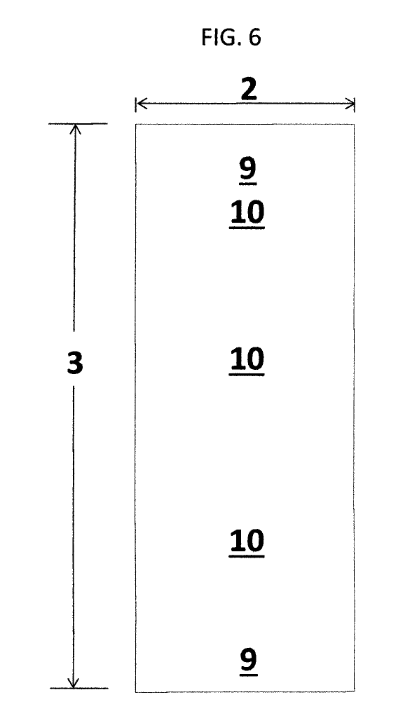

Notes, ride and or attraction heights, trademarks, copyrights, logos, sales and marketing information can be displayed on top of the background picture, image and or art. They should be placed on the poster so as not to obscure the straight edge ruler(s) and in a location that makes the poster aesthetically pleasing, see FIG. 1, FIG. 2, FIG. 5 and FIG. 6 Reference Number 9. There are no restrictions with the location of the items associated with Reference Number 9, although thought should be given not to cover the straight edge ruler(s) and to make the poster aesthetically pleasing. Font size and style for the straight edge ruler(s), ride and or attraction heights, trademarks, copyrights, logos, sales and marketing information are variable according to the poster size and purpose.

Reference Number 4 FIG. 1, FIG. 2 and FIG. 4 shows how ride(s) or attraction(s) could be annotated. There are no restrictions on how a ride(s) or attractions) can be annotated, although placement should coincide with the height requirement and the straight edge ruler measurement on the poster.

FIG. 1, FIG. 2, FIG, 4, FIG. 5 and FIG. 6 show the width, Reference Number 2, FIG. 1, FIG. 2, FIG. 3, FIG. 4, FIG. 5 and FIG. 6 show the height, Reference Number 3, of a Ride Stick Poster. The Ride Stick Poster may vary in height from 12 to 84 inches (30 to 213 centimeters), and vary in width from 3 to 50 inches (7.5 to 27 centimeters). The preferable dimensions are 8 inches (20.3 centimeters) wide and 52 inches (132 centimeters) tall. The straight edge ruler(s), on top of the background picture, image and or art do not have to start at zero (0). For example the measurement at the bottom of the poster may start at 24 inches (61 centimeters) and at the top of the poster end at 76 inches (193 centimeters). Another example could be if a horizontal straight edge ruler is used, the center of the poster could be zero (0) with the measurements increasing on both sides of zero (0).

On the back of the poster, FIG. 5 and FIG. 6, are the instructions for how to hang or display the poster, see Reference Number 10. There are no restrictions with the location of the items associated with Reference Number 10 on the back of the poster. Also, on the back of the poster can be notes, ride and or attraction heights, trademarks, copyrights, logos, sales and marketing information, and other information suitable to the poster's purpose, see Reference Number 9. There are no restrictions with the location of the items associated with Reference Number 9 on the back of the poster.

1. The Ride Stick Poster combines a picture, image and or art with a straight edge ruler and notes for the height requirements of various ride(s) and or attraction(s) for a particular amusement park, theme park or fair.

2. As in claim 1, the poster backing material can be made from any physical material or materials. The backing material(s) may vary in thickness from 0.0005 to 1.5 inches (0.012 to 38 millimeters).

3. As in claim 1, the poster may vary in height from 12 to 84 inches (30 to 213 centimeters), and the width may vary from 3 to 50 inches (7.5 to 127 centimeters).

4. As in claim 1, the printed or displayed picture, image or art, that is printed or displayed on the poster backing material is not limited.

5. As in claim 1, the straight edge ruler(s) and the notes, ride(s) or attraction(s) heights, trademarks, copyrights, logos, advertisements, sales, marketing or other information are printed or displayed on top of the background picture, image and or art. Placement of the ride(s) and or attraction(s) height notes should coincide with the height requirement and the straight edge ruler measurement on the poster.

6. As in claim 1, font size, style and height notation are variable according to poster size and purpose.

7. As in claim 1, the color of the straight edge measurements and ride and or attraction labels are not limited, contrast is required for legibility.

8. As in claim 1, the straight edge measurement can be in any measurement units, typically the units are in inches and or centimeters.

9. As in claim 1, the measurement does not have to start at zero, for example, the the poster measurement can range from 24 inches (61 centimeters) at the bottom to 72 inches (183 centimeters) at the top.

10. As in claim 1, on the back of the poster are the instructions on how to hang or display the poster, as well as, notes, the ride and or attraction height(s), trademarks, copyrights, logos, advertisements, sales, marketing or other information that are useful for the posters purpose.
